(19) United States
(12) Patent Application Publication
(10) Pub. No.: US 2019/0307839 A1
(43) Pub. Date: Oct. 10, 2019

(54) Enzyme Replacement Therapy for Melanoma

(71) Applicants: Pedro Brito Correia, Estoril (PT); Vincente Arez Teixeira, Lisbon (PT)

(72) Inventors: Pedro Brito Correia, Estoril (PT); Vincente Arez Teixeira, Lisbon (PT)

(21) Appl. No.: 15/947,821

(22) Filed: Apr. 8, 2018

(51) Int. Cl. A61K 38/17; A61K 9/00; A61P 35/00; A61K 38/16

(52) U.S. Cl. CPC A61K 38/17; A61K 9/0019; A61K 2300/00; A61K 38/164; A61P 35/00

(57) ABSTRACT

The most frequent cause of melanoma is Ultraviolet Radiation, which produces defects in DNA namely dimerisation of nucleotids in the same chain. There are in nature cases of particular resistance to UV radiation, namely the fungus Schizosaccharomyces Pombe, and resistance to oxidativ stress and gama radiation, namely the bacteria Deinococcus Radiodurans .

S. Pombe has a gene UVDE producing the protein UVDE—Ultra violet endonuclease, which is very interesting to avoid UV damage as occurring in melanoma.

Deinococcus Radiodurans produces the enzyme SOD2, a superoxide dismutase containing manganese 2+, which is a protein healing the errors caused by ROS—reactive oxygen species, which causes several oncological diseases and causes ageing.

These 2 proteins trigger the activity of other enzymes, namely ATM, CHEK2, p53, wellknown for participating in the correction of DNA errors, originated by several reasons,

The efficacy of this mixture of proteins is very improved if they are administered together with Cell Penetrating Peptides, which enhance the penetration of the membranes of individual cells but also crossing the Blood Brain Barrier, which conditions neurological therapies for diseases caused by DNA errors located in the brain.

### 1. FIELD OF INVENTION

Oncology, Age, Parkinson disease, Alzheimer disease, DNA replication correction mechanism, correction of carbonylation of proteins, repairing damages of UV radiation

### 2. BACKGROUND OF THE INVENTION

Diseases caused by virus, bacteria or other foreign bodies, which can be detected through antigens by the imunitarian system, are eliminated by apoptosis of the cells, but only under the condition that the membranes of the limphatic cells B or fagocitic cells contain the protein with a partop which may be connected to the epitop of the corresponding antigen.

Other diseases are not associated with antigens, but caused by the replication of some genes of DNA with errors or caused by the introduction of errors in DNA or in proteins by many external factors, like ultra violet light or reactive oxygene species—ROS.

In some cases the cells have the ability to correct the errors in DNA or proteins. If this mechanism does not work, the genes containing the errors produce proteins which originate diseases like
cancer: pre oncogenes RAS, WNT, MYC, ERK, TRKA, BCR-ABL, DHH, SHH, IHH, NURR1, LIF, VEGF, NGF, BDNF, NT3, NT4
Parkinson: genes LRRK2, ATN, producing alfa synuclein
Alzheimer: genes APP, PSEN1 and PSEN2 which cause beta amyloide production.
Aging by accumulation of oxydations and destruction of telomeres

The discovery of the repair of replication errors in DNA was recognised as a major achievement by the award of the Nobel Prize for Chemistry in 2015 to Paul Modrich, Aziz Somar and Thomas Lindahl.

This discovery has to be considered as important as the discovery of the imunitary system and a real alternative in therapeutics.

Errors in genes may be treated by gene therapy, which consists in the introduction or withdrawal of genes in the cells, using virus vectors, CRIPR, RNA-interference, Agrobacterium (for plants only).

The first commercial gene therapy was approved in 2003 in China and is called Gendicine, but it was not approved in USA and EU. Gendicine introduces the gene p53 in the tumor cells using an adenovirus.

This therapy may have a side effect, which results from the fact that both healthy cells and some cells containing error prone DNA may be repaired. If cells containing error prone DNA are repaired in a way that no healthy cells are produced but other cells containing the errors of the mother cell, the disease is not eliminated.

The company Uniqura obtained the approval in USA and EU in 2012 for the gene therapy based on Glycera, but just one person used this therapy until 2018. Therefore, in 2018 Uniqure took this product out of the market.

Gene therapy is very complex and very expensive.

DNA errors may also be corrected by introducing in the cells enzymes which have the ability to correct DNA errors. This is the approach in the present patent application, Enzyme Replacement Therapy.

It is known that the proteins ATM, CHEK2 and p53 trigger the correction mechanism of genes containing errors or cause apoptosis, whenever the error is too large to be corrected. The correction consumes energy delivered by the conversion of ATP into ADP, and stops if there is no more ATP available

ATM are the initials for Ataxia Telangiectasia. This is a disease where the protein ATM does not exist and the replication errors of DNA are not corrected.

ATR is also important and it is a complex of ATM and RAD3.

ATM is a kynase, a family of proteins which has the capacity to phosphorylate other proteins, and by this chemical reaction supply the energy necessary for the conversion of the initial protein in a protein with more energy. Phosphorylation is the enzymatic form to make chemical reactions without heating, as it is usually necessary for chemistry in the laboratory. The family of kynases are produced in mitochondria.

ATM has a molecular structure which allows the formation of a cycle which embraces and scans the DNA for anomalies, in a similar way as the PCNA, which participates later in the repair.

ATM operates in 2 steps:
activates the protein complex MRN, which includes the proteins MRE11, RAD50, NBS1 (Nijmegen Breakage Syndrome). The proteins RAD50 and MRE11 cause the alignment of the two DNA chains during repair. Protein NBS1 participates together with telomerase in the repair of the telomeres, situated at the tops of each gene.
The activated complex MRN changes the conformation of ATM, which increases its affinity to the proteins CHEK2 and p53 which are activated.

The protein ATM is produced by the gene ATM, which is located in the chromosome 11. ATM is a dimer, which is inactive in healthy cells. In the cells containing an error in DNA, ATM phosphorilates itself, which causes the dissociation in the two monomers. The monomers become activated and interact with the protein CHEK2 (chromosome 22), which following activates the protein p53.

An error in the gene CHEK2 producing the protein CHEK2, causes the impossibility of CHEK2 to participate in the repair mechanism of DNA and causes cancer or neurologic diseases originated by genetic disorders.

The protein p53 controls if the cells should have a DNA repaired or should suffer apoptosis, whenever the repair is not possible.

Oncologic cells do not have this protein or have an error on it. These cells may have the gene p53, but it is not expressed into the corresponding protein.

As observed by Richard Peto, cancer is not proporcional to the number of cells of the body. It was found in 2015 that elephants and whales have 10 to 20 genes p53, while humans have just one. As a consequence, elephants and whales have a very low incidence of cancer. In fact, if there is an error in one p53 gene, another identical gene produces the same protein to trigger the repair of the genes.

The gene p53 is located in the chromosome 17. It produces 15 proteins which are isomorphic, and participate in the repair of DNA.

There are humans with no gene p53 at all. They have a high incidence of cancer, which constitutes the syndrome of Li Fraumeni.

Therefore, healthy cells must have the proteins necessary for repairing errors whenever they occur. If there are already too many errors, the cells suffer apoptosis.

In response to DNA damage, cells activate the sensor kinases ATM, ATR and DNA-PK, that in turn phosphorylate multiple downstream substrates, including the effector kinases CHEK1 and CHEK2, resulting in cell-cycle checkpoint initiation and/or apoptosis

Of the four intervenient ATR, ATM, CHEK1 or CHEK2, CHEK2 is the most commonly altered and approximately 50% of patients with CHEK2 alterations also carry defects in the p53 signaling pathway, such as TP53 mutation/loss or amplifications of MDM2/4, both well-known p53 inhibitors.

CHEK2 is known to be required for the p53-dependent apoptotic response to radiation. In order to analyze the role of p53 in CHEK2 mediated tumor suppression, the authors in reference 16 crossed Ntv-a Chk2^{-/-} mice with p53^{-/-} mice and noted that PDGF-induced gliomas arise with a similar latency in both genetic backgrounds. Furthermore, studies have been made suggesting that CHEK2 and p53 are epistatic in the suppression of glioma formation. Epistasis is the need for a group of genes to act and not one gene alone.

The G1/S checkpoint, which prevents cells from entering S phase, is predominantly regulated by p53 and is defective in ATM null cells. Despite its well-known function in ATM-dependent induced p53 activation, the role of CHEK2 in the G1/S checkpoint is still controversial.

CHEK2 is required for the maintenance of the G2/M checkpoint in gliomas, but not for its activation, either in vitro or in vivo.

ATM directly phosphorylates p53 on Ser-15 and Thr-68 on CHEK2, which, in turn, phosphorylates p53 on Ser-20, thereby helping to regulate the cell cycle and apoptosis. Thus, CHEK2 works as both a transducer acting in the ATM-CHEK2-p53 cascade and a candidate tumor suppressor. Indeed, CHEK2 mutations are found in some hereditary malignancies, such as Li-Fraumeni Syndrome.

This repair mechanism will not start in the case of melanoma, where the error caused by UV radiation is the cyclobutane pyrimidine in DNA. This error causes a knick, which however is not detected in the scanning made by ATM or later PCNA.

Melanoma is 13 times less frequent in humans with black skin than in humans with white skin. This difference has been attributed to the existence of black melamine, called eumelamine in black skinned humans, while the white skinned humans have another kind of melanine called pheomelanine, which is yellow or brown.

In fact, the visible and UV absorption spectrum of eumelanine and pheomelanine present a difference of only 10% under 400 nanometer, UV wave lengths, but present 100% difference in the spectrum above 400 nanometer, visible wave lengths. This means that eumelanine is black because it absorbs in the visible range. Therefore, the UV protection is similar for black skinned and white skinned humans.

However, black skinned humans have in their skin 3 times more macrophages and capillaries of the lymphatic system, than white skinned humans. As a consequence, the number of apoptosis of cells containing UV damage is 8 times bigger in black skinned humans than in white skinned.

We consider that it is far better to correct mistakes in DNA as they appear than to wait the multiplication of the cells containing errors and later killing all of them. Killing cells is the goal of the imunitary system, while correcting errors is the goal of the DNA correction mechanism.

Proteins are lipophylic and have difficulty to cross cell membranes, which are lipophylic. One alternative to solve this difficulty could be to introduce across the cell membranes the plasmids generating the 5 proteins which we are claiming.

On the other side, it is not enough to produce the plasmids containing the genes corresponding to our 5 referred proteins, but is necessary to introduce also the proteins or further genes to start the translation of the plasmids.

As referred in our literature positions 17 to 21, both proteins and plasmids have difficulty in crossing the membrane of the cells without the help of CPP—cell penetrating peptides. These peptides were first discovered 20 years ago and change the cell membrane in a way that a cargo of larger molecules like proteins or plasmids cross the cell membrane together with the CPP.

The CPP can be covalently bound to the cargo or ionically. The alternative of covalent bonds has the inconvenient of secondary reactions.

CPP are currently used both in medicine as in cosmetics.

### 3. SPECIFICATION OF THE INVENTION

In order to make possible a repair by the ATM, CHEK2, p53 triggered mechanism, it is necessary to prepare the DNA or proteins, by a specific mechanism. Therefore we studied the extreme resistance of unicelular organisms to oxydative aggression or UV aggression.

The fungus Schizosaccharomyces Pombe, which is well known for its resistance to UV radiation, creates a knick in the cyclobutane pyrimidine errors, allowing the correction triggered by ATM, CHEK2 and p53 to localize the error and to correct it. The knick creates a geometric form which is detectable by scanning the DNA chain by ATM and later PCNA.
S Pombe has this ability due to the production of the protein UVDE—Ultra Violet Endo Nuclease. This enzyme contains 3 manganese 2+ ions, like the enzyme of Deinococcus Radiodurans SOD2.
UV light not only produces cyclobutane pyrimidine errors but also oxidative damage both in proteins and in DNA. Proteins are carbonylated in the amino acids containing an aromatic side chain.
Cancer and other genetic controlled diseases as well as aging are also caused by ROS—reactive oxygen species.
The ROS can attack both DNA and proteins. If the repair proteins react with ROS they loose their ability to repair DNA. The result of oxidation of DNA or repair proteins is therefore the same.
The best known enzyme to detect and correct these errors exists in the Deinococcus Radiodurans, which is also the most resistant known bacteria concerning gama radiation and the corresponding double chain breaks in DNA. The molecule which gives this resistance to Deinococcus Radiodurans is SOD2. In cases where the errors in DNA include cyclobutane pyrimidine or are caused by ROS, we found that the introduction in the blood circulation of the 5 proteins as proposed, allow an important progress in the treatment.

We consider that a protein therapy is better than a gene therapy for diseases caused by errors in gene, because:
proteins introduced in the cells are removed after a certain time by mechanisms existing in the cell based on ubiquitin, while genes can hardly be removed by a natural way, not using laboratory processes like CRISPR.
The permanence of artificially introduced genes may cause undesirable side effects.

The package of proteins to use in the therapy can be selected in order to include not only ATM, CHEK2 and p53 but also some important proteins which are eventually missing in the humans.

Therefore, we concluded the advantage of the intravenous local injection of a plasma solution of the proteins ATM, CHEK2 and p53 in order to supply the means for the activation of the repair mechanism, together with UVDE and SOD2.

In order to improve the efficacy of the 5 indicated proteins, we used one or more cell penetrating peptids, namely Tat, Pep1, MPG, and the arginine nonapetids SR9, HR9, PR9.

### EXAMPLE

The hair of 12 Rats was removed and a melanoma was induced. They are separated in 3 groups of 4 rats.

The first group received one weekly injection of a 1 ml solution containing plasma and 1 microgram of each protein ATM, CHK2, p53, UVDE, SOD2.

The second group of 4 rats received one weekly injection of a 1 ml solution containing plasma and 1 microgram of each protein ATM, CHK2 , p53, UVDE, SOD2 and 2 microgram of the CPP arginine nonapeptide SR9.

The third group received no injection.

After 2 months the melanoma of these rats is compared by immunocytochemistry The results are for group 1 a decrease of 15%, for group 2 a decrease of 75% and for group 3 an increase of 5% melanoma.

### 3. SPECIFICATION OF THE INVENTION

In order to make possible a repair by the ATM, CHEK2, p53 triggered mechanism, it is necessary to prepare the DNA or proteins, by a specific mechanism. Therefore we studied the extreme resistance of unicelular organisms to oxydative aggression or UV aggression.

The fungus Schizosaccharomyces Pombe, which is well known for its resistance to UV radiation, detects the knick in DNA double hélix caused by cyclobutane dipyrimidine errors, allowing the correction triggered by ATM, CHEK2 and p53 to localize the error and to correct it.

S Pombe has this ability due to the production of the protein UVDE—Ultra Violet Endo Nuclease. This enzyme contains 3 manganese 2+ ions, like the enzyme of Deinococcus Radiodurans SOD2.

UV light not only produces cyclobutane dipyrimidine errors but also catalyses oxidative damage both in proteins and in DNA. Proteins are carbonylated in the amino acids containing an aromatic side chain.

Cancer and other genetic controlled diseases as well as aging are also caused by ROS—reactive oxygen species.

The ROS can attack both DNA and proteins. If the repair proteins react with ROS they loose their ability to repair DNA. The result of oxidation of DNA or of oxidation of repair proteins is therefore the same.

The best known enzyme to detect and correct these errors exists in the Deinococcus Radiodurans, which is also the most resistant known bacteria concerning gama radiation and the corresponding double chain breaks in DNA.

The molecule which gives this resistance to Deinococcus Radiodurans is SOD2. In cases where the errors in DNA include cyclobutane pyrimidine or are caused by ROS, we found that the introduction in the blood circulation of the 5 proteins as proposed, allow an important progress in the treatment.

We consider that a protein therapy is better than a gene therapy for diseases caused by errors in gene, because:
proteins introduced in the cells are removed after a certain time by mechanisms existing in the cell based on ubiquitin, while genes can hardly be removed by a natural way, not using laboratory processes like CRISPR.
The permanence of artificially introduced genes may cause undesirable side effects.

The package of proteins to use in the therapy can be selected in order to include not only ATM, CHEK2 and p53 but also some important proteins which are eventually missing in the humans.

Therefore, we concluded the advantage of the intravenous local injection of a plasma solution of the proteins ATM, CHEK2 and p53 in order to supply the means for the activation of the repair mechanism, together with UVDE and SOD2.

In order to improve the efficacy of the 5 indicated proteins, we used one or more cell penetrating peptids, namely Tat, Pep1, MPG, and the arginine nonapetids SR9, R9, PR9.

### EXAMPLE

The hair of 12 Rats was removed and a melanoma was induced. The rats were separated in 3 groups of 4 rats.

The first group received one weekly injection of a 1 ml solution containing plasma and 1 microgram of each protein ATM, CHK2, p53, UVDE, SOD2.

The second group of 4 rats received one weekly injection of a 1 ml solution containing plasma and 1 microgram of each protein ATM, CHK2 , p53, UVDE, SOD2 and 2 microgram of the CPP arginine nonapeptide SR9.

The third group received no injection.

After 2 months the melanoma of these rats is compared by immunocytochemistry The results are for group 1 a decrease of 15%, for group 2 a decrease of 75% and for group 3 an increase of 5% melanoma.

1. We claim an enzyme replacement therapeutic process for melanoma using in each application a 2 milliliter plasma solution of the 5 following proteins: 10 micrograms of UVDE, which detects errors in DNA caused by Ultra Violet light, 10 micrograms of SOD2, which avoids both in enzymes as well as in DNA errors caused by reactive oxygen species, proteins 5 micrograms ATM, 5 micro grams of CHK2, and 5 micrograms of p53, which trigger the correction mechanism of DNA in cells, where these 5 proteins are produced by well-known monoclonal antibody technology, whereby the plasma solution is injected in the blood of patients suffering from melanoma, whereby these proteins are injected together with cellular penetrating peptides in order to increase substantially the possibility of the proteins crossing the cell membranes as well as the Brain Blood Barrier.

2. In the process of claim 1 where the therapy is adapted by adding or withdrawing any protein according to the mechanism of gene or protein repair designed for the individual patient.

3. In the process of claim 1 where the quantity to be administered may vary between 1 and 10 microgram per treatment and per protein according to the weight of the person and the reaction to the first administered quantities.

4. In the process of claim 1 where the CPP—Cell Penetrating Peptides are Tat—Transactivator of Transcription, Pep1, MPG, as well as Arginine nonapetides SR9, HR9, PR9, whereby these indicated CPP do not exclude the application of similar compounds and whereby in the case of Tat protein a quantity of 7 micrograms per millilitre are to be used.
